(19) United States
(12) Patent Application Publication
(10) Pub. No.: US 2015/0122263 A1
(43) Pub. Date: May 7, 2015

(54) CHEEK DEPRESSORS AND METHOD OF USE

(71) Applicant: James P. Viken, Eden Prairie, MN (US)

(72) Inventor: James P. Viken, Eden Prairie, MN (US)

(21) Appl. No.: 14/070,910

(22) Filed: Nov. 4, 2013

(51) Int. Cl. A61M 16/06

(52) U.S. Cl. CPC A61M 16/0605; A61M 16/0688; A61M 2016/0661; A61M 2210/0637
USPC 128/206.25; 128/206.24; 128/206.26

(57) ABSTRACT

A device and method of use for depressing the cheeks of a user, to limit significant motion of the cheeks and provide for enhanced inhalation and exhalation. The device may be used to enhance respiration during a variety of activities, and is especially beneficial during the use of positive airway pressure (PAP) type medical. devices The device may include a pad adapted to engage a target area of a user's cheek and to maintain cheek tissue against a bone surface during inhalation and exhalation. The device may also include at least one support element coupled to the pad to maintain the pad at the target area during a therapy session.

### RELATED APPLICATIONS

This application claims the benefit of U.S. application Ser. No. 12/945,826, filed Nov. 12, 2010, which claimed the benefit of U.S. Provisional Application No, 61/260,682, filed Nov. 12, 2009, and each application being incorporated herein by reference.

### FIELD OF THE INVENTION

This invention generally relates to a user interface device and more particularly to a device for enhancing respiration during various user activities.

### BACKGROUND OF THE INVENTION

A positive air pressure (PAP) type respiration system can be used with a face mask type device to deliver pressurized air to the patient's airway. This pressurized air is delivered via various types of face masks, a nasal mask, a nasal and mouth hybrid type mask, or a full face mask. There are a number of nasal masks such as a “nasal pillow” mask and the like, as well as other newer designs for masks such as nasal prong or oral masks. A PAP type system typically includes an air delivery hose which is connected between a PAP air source and a mask which is typically custom-fitted to the patient's face or part of the face being used.

When using a PAP type device, if the patient desires to use a nasal mask or a hybrid type mask then usually a chin strap is required to keep the mouth closed, Use of either of these two types of mask devices without a chin strap device can allow pressurized air to escape from the mouth, disrupting adequate air delivery to the lungs for effective ventilation.

Motion of the cheeks inward and outward can occur during certain physical exercise, during use of a PAP type device, or during the playing of an exhalation-powered musical instrument,

This cheek motion can create a variability of the inner volume of the mouth cavity and cause an air accumulator effect to occur, thus making inhalation and/or exhalation less direct and efficient. This air accumulator effect can make the automatic controls of a PAP type device less sensitive, and can result in the use of higher air delivery pressure than would otherwise occur. Unnecessarily higher air delivery pressures in a PAP type device can make the use of a PAP type device less comfortable and decrease patient compliance with the prescribed treatment. Cheeks expanded by air pressure inside the oral cavity and significant cheek motion can he judged unpleasant by PAP type device users. Therefore a need exists to limit significant cheek motion during use of a PAP type device or during certain physical activity.

Obstructive Sleep Apnea (OSA) and other sleep disorders can contribute to the onset of other serious chronic health problems such as high blood pressure, obesity, heart disorders, diminished concentration, excessive tiredness during the day, failing asleep while driving, and depression and anxiety disorders. Untreated OSA and other sleep disorders may contribute to serious health disorders such as autoimmune disorders and even cancer. USA and other sleep disorders which are untreated or inadequately treated can reduce one's quality of life and even seriously damage one's health over time and may contribute to a significantly shorter life expectancy.

PAP devices have been developed to be automatically adjusting to detect and limit air pressure delivery required to stop the occurrence of USA, thereby making the operation of the devices more comfortable for the patient and thereby increasing the likelihood of patient compliance. Sensors have been used to determine if USA or other sleep disorders are occurring due to inadequate pressure being supplied to the air from the PAP type device for good ventilation, and automatic controls have been invented and developed for increasing the pressure of the air delivered until such USA type and other sleep disorder events are reduced or stopped.

The prior art includes Australian Patent Application No. AU2005100738, to Respironics, entitled Patient Interface Device. Disclosed therein is a patient interface device which includes a frame that is sized and configured to span at least a portion of the patient's face while remaining below the patient's eyes when the patient interface device is worn by the patient. The patient interface device includes a support member that is sized and configured to span at least a portion of a patient's face while remaining below the patient's eyes. The patient

interface device purportedly provides a stable platform that supports a sealing assembly on the patient, while minimizing the amount of material worn on the patient's face and head.

### SUMMARY OF THE INVENTION

Cheek depressor (CD) devices of the present invention engage a targeted region of a patient cheeks with a focused application of external forces at the targeted regions to limit significant cheek motion during inhalation and exhalation and with substantially no external forces being applied to the user's zygomatic bone or mandible by the CD devices. The target area is located between the mandible and zygomatic bone of the user.

The CD device enhances the effectiveness of PAP type medical device, enhances respiration during exercise or pursuit of individual or team sports, and enhances respiration while at rest or anytime and during any leisure activity. The CD device's specific size, shape, including shape of its inner cheek facing surface, and the three used to press it against each cheek can be customized to the individual user and the specific application. The CD device can be used in combination with PAP type devices and other applications where it is helpful to enhance respiration by preventing significant cheek motion which can occur during any exercise, including sport activities and playing of musical instruments,

Typically the CD device is pressed and held in place against the cheek at the target area with sufficient force to move the cheek inward to make contact with the teeth, gums and or upper or lower jaw, e.g., maxilla surfaces, but not so much force to abrade, bruise or harm the teeth, gums or upper or lower jaw or the jaw joint. The use of the CD device can prove useful by enhancing inhalation or exhalation with any activity which can produce significant cheek motion, including the forceful playing of exhalation powered musical instruments where strength of breath and stamina are important considerations. When pressed against each cheek with adequate force necessary to limit significant cheek motion, the CD device may make respiration more efficient during physical exercise, especially strenuous physical exercise, and enhances respiration any time, including during non-exercise.

A cheek depressor device embodiment of the present invention offers a means of preventing significant cheek motion during inhalation or exhalation which reduces an air accumulator effect of the mouth cavity. Embodiments of the invention can be used to enhance respiration in many diverse applications, including while recreating or while using a PAP type device. Other useful applications in medicine, leisure and industry will become clear as these devices gain more use.

In one embodiment, a CD device engages a target area of the cheek in order to enhance inhalation or exhalation by preventing significant cheek motion. In order for the device to be maximally effective it should be positioned to depress the targeted area of the cheek toward the inside of the mouth to cause the cheek to make contact with the teeth and gums. The device should be large enough and have its inner cheek side configured in a curved or slope manner such that it is able to provide the necessary contact with the cheek in order to prevent significant cheek motion during inhalation or exhalation.

The CD device can be used to enhance respiration, including, but not limited to, during the use of medical equipment such as positive airway pressure (PAP) type devices, or during the pursuit of sports. It can also be used to assist in the playing of exhalation powered musical instruments. The invention can be used to increase the effectiveness of the medical use of PAP type medical devices, including those which are non-automatic such as a basic CPAP device, as well as those that are automatically self adjusting such as an automatic CPAP, BiPAP, Bi-Level, V-Pap, C-Flex, A-Flex or the like which are typically used to treat Obstructive Sleep Apnea (OSA) and other sleep disorders, and which automatically adjust their air delivery pressure based on the patient's response, as indicated by sensors and processed by complex algorithms and microprocessors.

One object of this invention is to increase patient comfort as a means of increasing patient compliance when used with a PAP medical device. It is well known in the art that patient compliance with a PAP type device is reduced by a patient's discomfort while using that PAP type device. Therefore establishing and maintaining maximum patient comfort during the use of the PAP type device is an important and key factor in determining patient compliance.

Any significant motion of the cheeks during use of a PAP type device can be perceived by some patients as uncomfortable when it becomes noticeable and this is a common occurrence if higher air delivery pressures are used, or such higher air pressures occur as a response of any automatic, self-adjusting controls of any PAP type device.

Unrestricted cheek movement during use of a PAP type device can create a hydraulic accumulator effect which can reduce the effectiveness of the pressure transmission of the PAP device resulting in delayed pressure transmission, This delay of pressure transmission to the airway can necessitate higher PAP pressure settings than would otherwise be required to maintain adequate ventilation of the lungs. The accumulator effect can therefore increase the effort of the lungs needed to produce exhalation. Therefore, use of the CD device can decrease the effort of the lungs to produce inhalation and exhalation and may allow the use of lower settings of the PAP type device, enhancing comfort and compliance with the prescribed treatment.

Significant cheek motion during use of a PAP device itself is judged uncomfortable in some patients, and preventing cheek motion can contribute to patient comfort during their use of a PAP type device. Use of the CD device to depress the approximate center of the cheek can decrease the effort required to exhale by significantly preventing the accumulator effect that is associated with significant cheek motion, and can thereby increase the comfort of the patient while helping to elicit a quicker more direct response in any automatic, self-adjusting controls that the PAP type device may have, helping to keep the pressure of the air delivered by the PAP type device to a minimum level by reducing the amount of OSA or sleep disorder events.

An important object of the CD device is to increase the responsiveness of the automatic self-adjusting PAP device's control systems to allow a lower air delivery pressure setting than otherwise might be possible. This can help to increase patient comfort if the pressure can he somewhat reduced while still being adequate to prevent the occurrence of OSA or other sleep disorder events. It is known in the art that the higher the air delivery pressure by the PAP device, the greater the tendency for air to leak around the mask, whether it be a nose only mask, a full face mask or a hybrid form of the two. When air leaks occur due to higher air delivery pressures being used, then the solution typically is the tightening of the straps or headgear used to hold the mask type device securely to the face. It is known that tightening of the straps can decrease patient comfort in some cases.

During the use of a PAP type device, holding the air delivery pressure down to the lowest possible level while still high enough to prevent the occurrence of OSA is an important consideration for PAP and mask device designers. Air teaks through the mask device, which typically occur at the edge seals of the full face type masks, can generate annoying noise which can wake the patient or his or her sleeping partner. When using an automatic self-adjusting PAP type device, these air leaks through the edge seals of the mask will typically cause the device to automatically self-adjust to a higher air delivery pressure output to compensate for the leakage and pressure reduction. The use of the CD device can result in a barrier provided against the side seals of the full face mask and can help prevent these unwanted air leaks which can be annoying and cause an unwanted increase in the pressure output of the PAP type device.

Therefore, another important object of the use of this invention is to reduce the air leakage of the side seals of a full face mask during the use of a PAP type device by arranging or positioning the invention to make contact with or to reinforce those seals, thereby helping to keep those seals snug against the front sides of the cheeks, and/or serving as a barrier to prevent those seats from being displaced outward and producing unwanted leakage of air from the mask.

Another object of the CD device is to provide an inexpensive, easy to manufacture means to prevent significant cheek motion in many different activities including but not limited to recreation or during the use of a PAP type devices.

Another object of the invention is to provide a relatively inexpensive, easy to manufacture, easy to wear and use device which can be made integral to a PAP type device mask, chin strap or headgear, or easily installed on a headgear or chin strap for use with a PAP type device, or which can be made to he a free standing support structure similar to a pair of ear phones but positioned at the approximate center of the cheeks for many different uses, thereby providing a useful invention in many different forms which are simple and cheap to manufacture, thereby contributing to less expensive manufacturing in order to reduce carbon emissions that would otherwise be higher.

Another object of the present invention is a CD that depresses the cheek at the target area to minimize or prevent an air accumulator effect within the oral pharygeal cavity in order to enhance breathing without abrading, bruising or harming the inner cheek, gums, or teeth, especially when using a PAP type unit or engaging in activities which involve hard exhalation, thereby keeping the volume of the oral pharyngeal cavity minimal during respiration, especially during exhalation which would otherwise displace the cheeks outward and expand the oral pharygeal cavity.

Other purposes will appear in the ensuing specification, drawings and claims. The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 is a side elevational view of an embodiment of a cheek depressor of the present invention.

FIG. 2 illustrates a target area for positioning of the device of FIG. 1

FIG. 3 is a perspective view of the cheek depressor of FIG. 1.

FIG. 4 is a portion of the cheek depressor of FIG. 1.

FIG. 5 is a side elevational view of a second embodiment of a cheek depressor of the present invention.

FIG. 6 is a side elevational view of a spring and pad portion of the embodiment of FIG. 5.

FIG. 7 is a side elevational view of a third embodiment of a cheek depressor,

FIG. 8 is a spring and pad portion of the embodiment of FIG. 7.

FIG. 9 illustrates a side elevational view of a fourth embodiment of a cheek depressor of the present invention.

FIG. 10 is a side elevational view of a fifth embodiment of a cheek depressor of the present invention.

FIG. 11 is a spring and pad portion of the embodiment of FIG. 10.

FIG. 12 is a side elevational view of a sixth embodiment of a cheek depressor device of the present invention.

FIG. 13 is a side elevational view of a pad portion of the device of FIG. 12.

FIG. 14 is a side elevational view of the pad portion of FIG. 11.

FIG. 15 is a side elevational view of a pad portion suitable for use with the device of FIG. 12.

FIG. 16 is a side elevational view of a pad portion suitable for use with the device of FIG. 12.

FIG. 17 is a view of a seventh embodiment of a cheek depressor of the present invention.

FIG. 18 is another view of the cheek depressor of FIG. 17.

FIG. 19 is another embodiment of the cheek depressor of FIG. 17.

### DETAILED DESCRIPTION OF THE INVENTION

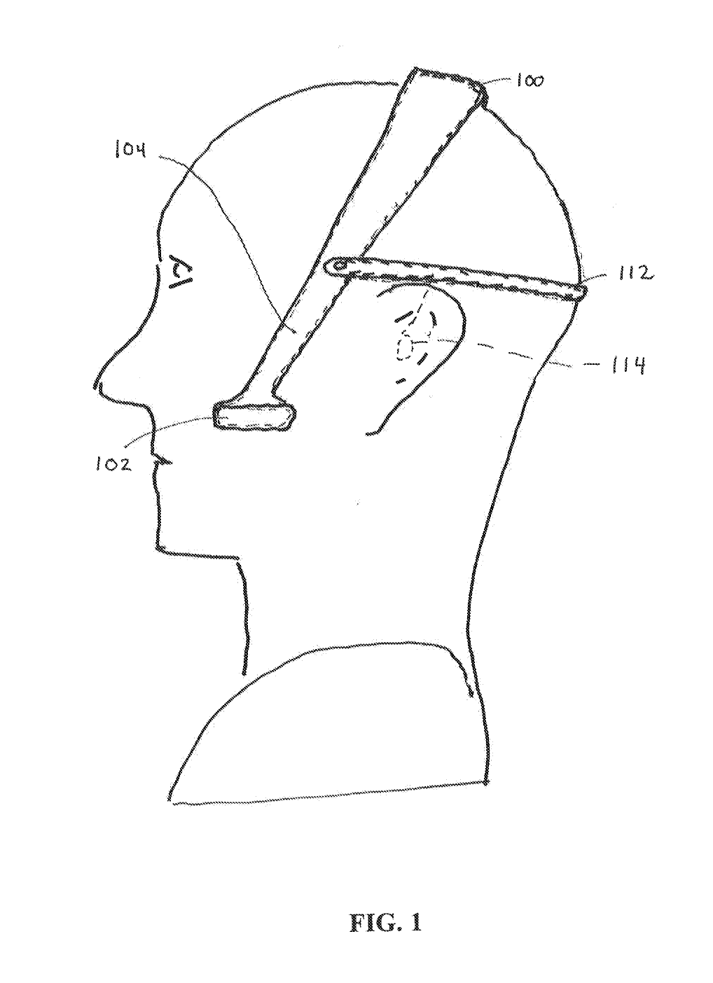

FIG. 1 depicts another embodiment of the invention in a free standing, yoke supported form, identified as CD assembly 100. CD assembly 100 is a free standing unit that is comprised of a spring yoke 100 for support and placement of a pad 102 used in a pair, one affixed to each lower end of spring yoke 104.

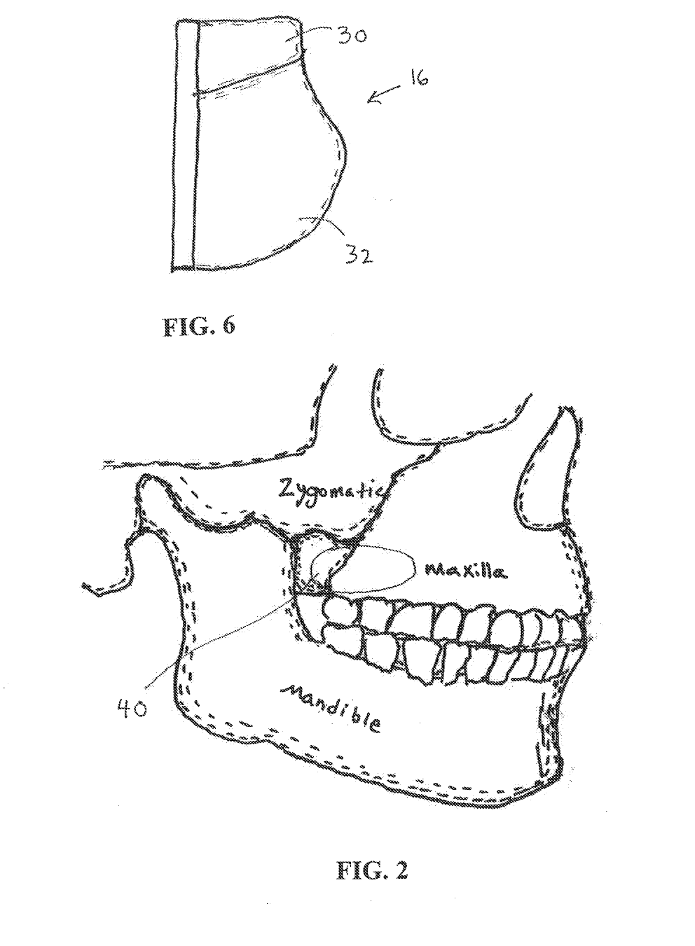

Referring to FIG. 2, the target area is defined as region 40, namely that part of the cheek where the lower lateral pterygoid process bone intersects with a maxilla bone and a zygomatic bone (also called the zygomatic arch or cheek bone). The target zone is preferably centered between the zygomatic bone and the upper teeth. The infratemporal surface of the maxilla is contained in the target zone. The posterior superior alveolar nerves cross through the target zone. The CD devices of the present invention preferably provide a force of between about 0.5 lbs-f to 3 lbs-f to the target areas on each side of the user's head. Farces in the range of about 0.05-1.5 lbs-f are delivered to the target areas via various pads of CD devices of the present invention. These external forces applied to the target area collapse the check tissue against the teeth surfaces of the user. Substantially no external force is applied by the CD device to the zygomatic bone or mandible of the user. Minimal inwardly directed forces may be applied against the lower teeth, however, substantially no external force is applied toward moving the mandible forward or rearward.

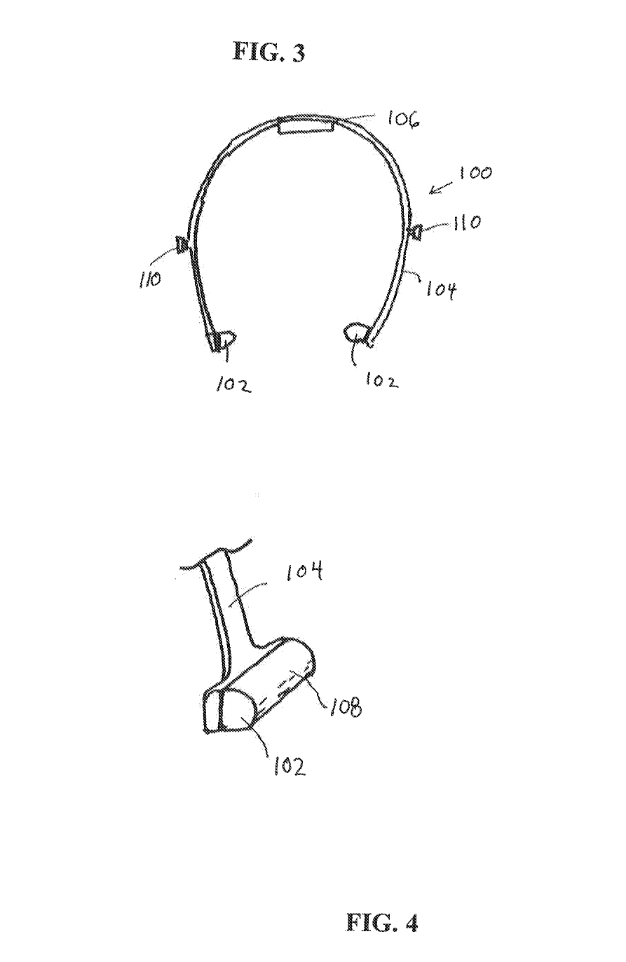

As shown in FIG. 3, a top pad 106 is affixed to spring yoke 104 for user comfort and positioning. Pad 106 is compressible with rebound memory such that it can provide and apply upward force to spring yoke 100. Different thicknesses of pad 106 and different rates of compressibility can be used in order to provide different levels of pressure to the target area while CD assembly 100 is properly arranged and positioned on a user.

As shown in FIG. 4, pad 102 has a pad surface 108 which is convex. Double sided sticky tape (not shown) can be applied to the convex pad surface 108 to help keep pad 102 in proper position while arranged and positioned on the head of a user. This tape is available as a medical grade tape and has uses in affixing medical and hair appliances. Alternatively medical grade glue can be applied to the convex surface of pad surface 108. In another embodiment, pad 102 can be manufactured with a surface comprised of a plastic or rubber like material which remains sticky but will not erode or transfer to the user, even after washing or using. These types of materials are available from several different manufacturers and can be used in the manufacture of pads like pad 102 to help keep CD assembly 100 in proper position during use, especially during hard physical exercise with a lot of physical movement and/or jarring.

Spring yoke 104 can be made in several different sizes such as small, medium, large, and extra large to accommodate different users with different sized heads, or can have telescoping adjustment means built into the lower ends of each side or anywhere on each side or at the top (not shown). Spring yoke 104 is provided with a knob 110 on each side, and the knobs 110 are for retaining a strap 112, which is shown in FIG. 1. Strap 112 is somewhat elastic and may be made of a rayon type strapping which has some rubber woven into it for mild elasticity, however many other choices are available for use in constructing strap 112 which would be suitable. Strap 112 is provided with an orifice at each end which is insertable over knob 110 to provide for retention of strap 112 in proper position when CD assembly 100 is arranged and positioned properly on the user as shown in FIG. 1.

CD assembly 100 is arranged and positioned on the head of a user as shown in FIG. 1, and with each of pads 102 arranged and positioned to make contact with the target area of the cheek of the user. Many different configurations for spring yoke 104 can be utilized, some of which include a spring yoke designed to fit from under the chin or to fit from behind the head. Alternatively a yoke can be integrated into a headphone unit or into almost any helmet design used in sports if such design takes into account safety needs and constructs the pair of pads 102 to be suitably collapsible or displaceable upon hard impact, especially for high contact sports. CD assembly 100 can be designed and constructed such that pad 102 can be made to be adjustable at the bottom ends of spring yoke 104, with such adjustment for angle to spring yoke 104, or to the target area, without departing from this novel art, Manufacturing CD assembly 100 with said adjustments can allow the user to specifically adjust each pad for the desired and best contact and pressure to be applied to the target area.

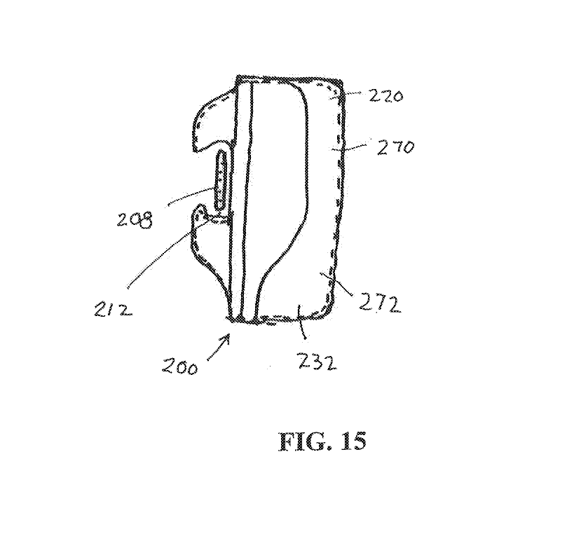

CD assembly 100 can be manufactured in many different configurations to fit the personal preference of different individual users. It can have a spring yoke configured and constructed which will allow it to be placed under the chin, or around the neck in back, or even placed in front of the head, above or below the nose. It can be constructed to contain ear phones for audio, enabling the user to listen to a radio, iPod or cell phone while using the CD assembly such as the type of CD assembly 100. In addition, a yoke can be constructed of a rigid material which holds its approximate form even when worn, and the spring action for asserting pressure against the target area can be supplied by the compressibility of the pad such as pad 102 or a spring system inside or behind it. The pad can be constructed in a larger combination form such as shown in FIG. 15, which will depress the approximate center of the cheek and the cheek area just below the zygomatic bone.

FIG. 1 shows strap 112 arranged and positioned to hold CD assembly 100 in proper position. The use of pad 106 (shown in FIG. 3) to apply pressure between the user and spring yoke 104, along with strap 112 and the pressure applied bilaterally to each cheek target area by the pair of pads 102, provides sufficient support to keep CD assembly 100 in position in most situations, including vigorous exercise. An additional strap (not shown) could he affixed to the pair of knobs 110 and arranged and positioned to cross the forehead of the user or even under the nose of the user in order to provide good support.

Of course, if the pair of pads 102 is provided with double sided medical grade adhesive or tape, then even more stability can be provided to CD assembly 100. If the CD assembly 100 is carefully fit with suitable pressure applied at pad 106 and suitable pressure applied at the bilateral pair of target areas of the cheeks of the user, then it is possible to construct CD assembly 100 without use of any strap(s) such as strap 112. Pad 102 itself can be constructed of a compressible foam plastic or rubber like material with rebound memory that can be arranged to exert sufficient and desired depression of the target area if a rigid yoke structure is utilized to provide support for the pair of pads used such as pad 102.

Spring yoke 104 can be shaped to provide some upward force from the horizontal to the target area, less than that applied horizontally to the target area In this embodiment an upward angle of about 35 degrees from the horizontal was used to vector the upward force into the target area. Other angles could be used including up to a vertical force, and this capability could be manufactured into the CD depressor assembly 100 allowing easy adjustment by the user. It was determined by the inventor during experimentation that CD assembly 100 works somewhat more effectively in some individuals when there is some upward angular force applied to the target area to press snugly against the lower end of the zygomatic bone and the lower side of the lower lateral pterygoid process bone while exerting slight inward pressure against the upper portion of the maxilla bone. These pressures should be sufficient to depress the target area of the cheek suitably, and also for optimal effectiveness can have some of the force applied at an upward inward angle from the horizontal, which can provide more enhancement of breathing in some, all without abrading, bruising or harming the inner side of the cheek, the gums or the teeth.

Spring yoke 104 is strong enough to assert adequate pressure to each of the bilaterally positioned pads 102 to keep the target area suitably and adequately depressed without abrading, bruising or harming the array of upper teeth, gums or inner cheek. In one embodiment, a force of approximately 1.20 lbs was applied through each pad 102. However individual needs can vary and adjustments can be designed, manufactured and incorporated into the finished product which will allow pressure to be adjusted to an increase or to a decrease within a fairly broad range, generally within 0.5 lb to 3 lbs, but individual cases could require less or more. Pad 106 can also be supplied in different thicknesses and hardnesses, if suitable means for adjusting length are not provided to CD assembly 100. In addition telescoping means can be provided to the top of spring yoke 104 to increase or decrease the pressure applied to each of the pair of pads 102.

CD assembly 100 is designed as a freestanding unit which can be used alone or with many mask assemblies for PAP usage. CD assembly 100 can be used alone to enhance breathing and respiration during exercise or sports, or anytime based on the personal preference of the user. It may also be helpful for some who play exhalation powered musical instruments by enhancing exhalation when it is used with the small pad shown in FIG. 1, which depresses the area of the cheek under the zygomatic bone.

When CD assembly is properly arranged and positioned such that the pair of pads 102 are suitably depressing the target area bilaterally on the cheeks and strap 112 is properly arranged and positioned to retain CD assembly 100 in proper position, breathing and respiration can be enhanced. The suitable and effective depression of the target area of the user's cheek enhances respiration in a significant number of individuals perhaps by neurological or neuromuscular stimulation means. FIG. 1 shows how CD assembly 100 appears when properly arranged and positioned whereby the pair of pads 102 are positioned to suitably depress the target area of the cheek. For maximum effectiveness in enhancing breathing and respiration, pressure must be applied to the target area in a sustained and focused fashion, but even minimal pressures appear to enhance breathing and respiration somewhat in a significant number of individuals.

A sustained and significant pressure to the target area appears to directly enhance inhalation as well as exhalation somewhat, even without depressing the approximate center of the cheek. And in addition, it was determined that if some pressure was applied to the target area in an upward angle from the horizontal, there was an additional enhancement of breathing in some individuals. CD assembly 100, when properly arranged and positioned, may enhance breathing and respiration during normal sleeping in some individuals, even those without Obstructive Sleep Apnea (OSA) or any diagnosable sleep disorder.

Sustained pressure provided to the cheek area right below the zygomatic bone can enhance breathing and respiration in some individuals. Thus the proper arranging and positioning of CD assembly can enhance breathing and respiration, and when the CD is applied to the approximate center of the cheek it can significantly reduce or substantially eliminate an air accumulator effect which can reduce the effectiveness of one's usage of a PAP device, it should be noted that sustained pressure to the cheek area just below the zygomatic bone appears to enhance breathing and respiration perhaps by neurological and/or neuromuscular stimulation.

For a significant number of individuals, sustained pressure properly applied to the target area can serve to enhance breathing and respiration as long as the sustained. pressure is applied to this area in an amount great enough to depress this area of the cheek deeply but not too great to abrade the inner cheek, or bruise the upper gums or harm the upper array of teeth. The amount of pressure needed to attain this for any given individual can vary and that must be kept in mind, and different thicknesses of pads can be made available if necessary or pads can be provided with different rebound rates.

The suitable depression of the target area by applied, sustained pressure can be attained by various structures represented in the illustrations or their equivalents, or in the structures disclosed but not shown specifically. For a significant number of individuals it is the sustained and focused application of pressure to this area of convergence of the lower lateral pterygoid process bone, the maxilla. bone and the zygomatic bone which allows the user to attain the maximum enhancement of breathing whether or not a particular embodiment disclosed, or an equivalent of that, depresses the approximate center of the cheek. For example if a Cheek Depressor is to be used during the use of a PAP type device or while playing an exhalation powered musical instrument, or while engaging in heavy exercise with a lot of movement and high exertion, a significant number of individuals will benefit from using an embodiment that emphasizes and focuses pressure to depress the target area of the cheek in addition to depressing the approximate center of the cheek. And for other situations where exercise is light, or there is no exercise involved, use of the Cheek Depressor in a free standing embodiment is an appropriate choice and will enhance breathing for a significant number of individuals. If an individual is engaging in an activity which requires and produces hard exhalation such as running or heavy exertion sports or playing an exhalation powered musical instrument, then it would be a good choice to use a Cheek Depressor embodiment which depresses the approximate center of the cheek and also focuses specific pressure on the target area.

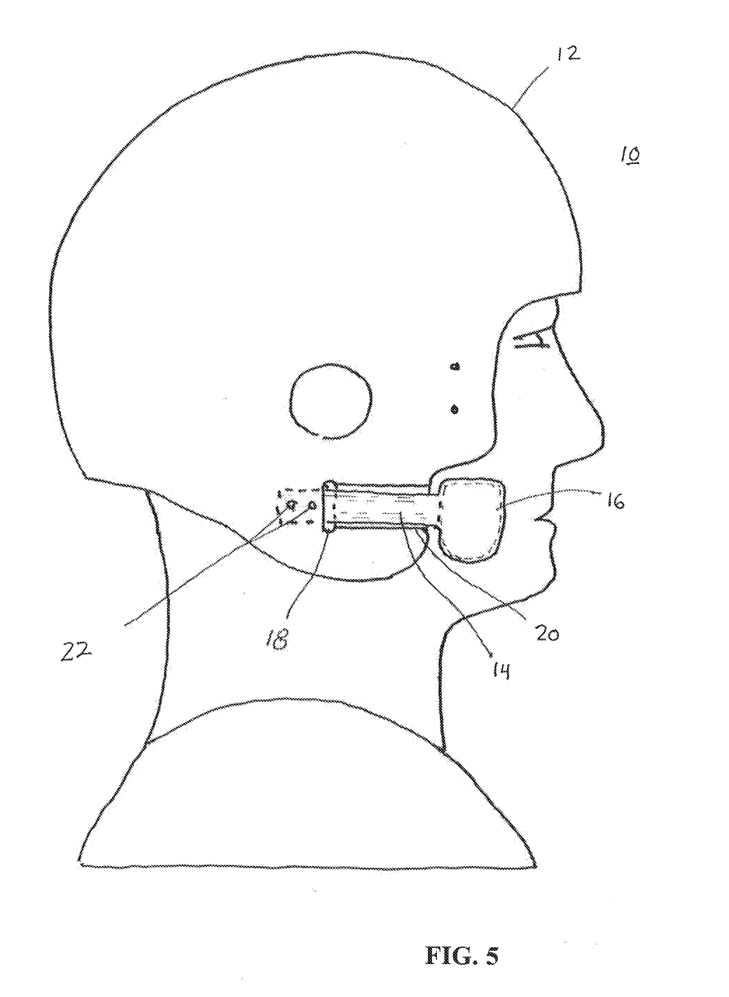

FIG. 5 is a side view illustration of an embodiment of the present invention. A sports helmet assembly 10 includes a shell 12, spring 14 and a check-engaging pad assembly 16 which is attached to spring 14. Helmet assembly 10 includes a padded shell of traditional construction. Spring 14 at one end passes through an aperture 18 of shell 12 and is then fastened to the inside of shell 12. A portion of spring 14 rests in a surface channel 20 in shell 12. As described herein, pad assembly 16 defines a cheek depressing device. Spring 14 is held onto shell 12 via fasteners 22, such as rivets, etc.

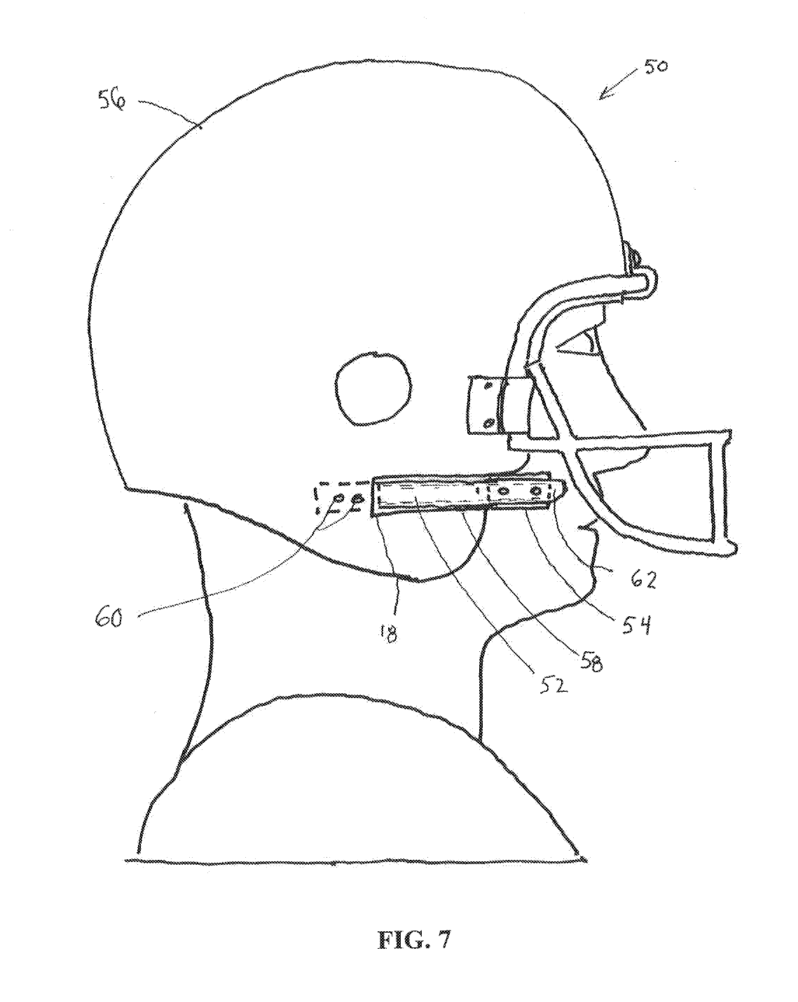

FIG. 6 illustrates pad assembly 16 as including two pad portions, a hard pad portion 30 and a soft pad portion 32 which are preferably molded together and affixed to spring 14. Soft pad portion 32 is made of a foam plastic material similar to foam rubber in its properties and is moderately compressible, whereas hard pad portion 30 is generally non-compressible. Soft pad portion 32 is thicker at its approximate center than hard pad portion 30. During use, pad assembly 16 is positioned onto the cheek of the user and is held against the cheek by spring 14. Hard pad portion 30 suitably depresses a target area of the cheek of the user, and soft pad portion 32 suitably depresses the approximate center of the cheek of the user with soft pad portion 32 being compressed by spring 14. Pad assembly 16 can be custom molded, shaped, and constructed specific to each user's cheek anatomy and hard pad portion 30 and soft pad portion 32 can be constructed within pad assembly 16 in a variety of different hardnesses and compressibility to provide for individual differences.

In order to put on or remove football helmet assembly 10, the user must pull springs 14 apart to move pad assembly 16 outward away from shell 12, after which the springs 14 can be slowly released once the helmet is donned. During hard shocks spring 14 will be able to flex somewhat thereby adjusting to the exact temporary position of the users face and head inside football helmet assembly 10. The simultaneous depression of the target area and the approximate center of each cheek can provide enhanced breathing.

A helmet assembly can be constructed with an integral cheek depressor in a variety of alternate ways. For example, a rigid plate which is shaped approximate to the size of the intended cheek depressor can be attached to the lower front edge of each side of the helmet closest to the chin of the user and this plate can be attached by use of a spring hinge which will apply the desired pressure to the specific pad design and size. A smaller plate may be used to hold a pad intended to depress the target area only, or a larger plate may be used to hold a pad. intended to depress the target area of the cheek and also the approximate center of the cheek. A combination pad assembly comprised of a hard pad and a soft more compressible pad may also be used.

FIG. 7 depicts another embodiment of the present invention. A football helmet assembly 50 includes a bilateral and integral cheek depressor comprised of a spring 52 and pad 54. Football helmet assembly 50 is comprised of a generally rigid shell 56 having a padded interior, Shell 56 is provided an aperture 18 through which spring 52 passes into and through. Shell 56 is also provided with a surface channel 58 deep enough to retain spring 52 at or beneath the outside surface of shell 56. When it is properly installed and in proper operating position after football helmet assembly 50 is in proper wearing position.

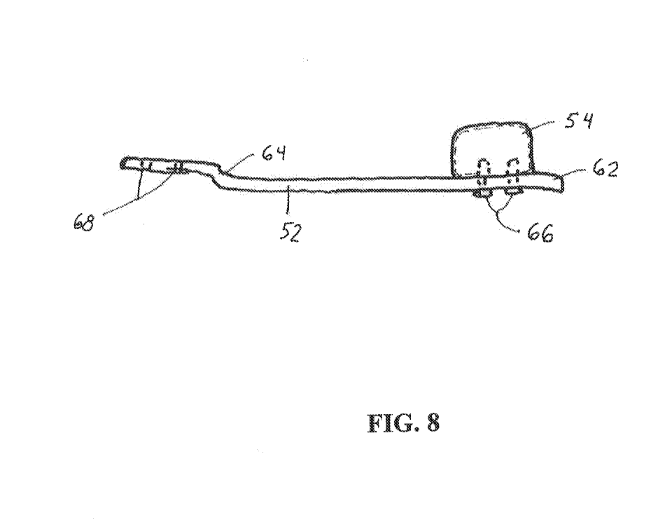

FIG. 8 shows spring 52 as being bent to allow it to be inserted through aperture 18 and to then engage against surface channel 58 after it has been secured in place, via for example a pair of rivets 60. Spring 52 is provided with a tab 62 at one end which allows the user to grasp spring 52 and pull it and its attached pad 54 away from shell 56 in order to either put on or remove football helmet assembly 50.

Spring 52 is provided with a pad 54 which is substantially firm and is molded, such as with a medical grade silicone compound. Pad 54 has a slight convex shape to its inner surface which faces the user. FIG. 8 shows spring 52 to have a double opposing bend 64 provided for spring 52 to fit properly into and through aperture 18. If the user desires a slightly upward force applied to the target area, a slight twist can be added to spring 52. A plurality of fasteners 66 are used to connect spring 52 to pad 54. Spring 52 includes a plurality of apertures 68 through which fasteners 60 are received during installation.

Football helmet assembly 50 of FIG. 7 provides an integral cheek depressor allowing a football player to enhance his breathing while playing football, During hard shocks spring 52 will be able to flex somewhat thereby adjusting to the exact momentary positional change of the user's face and head inside football helmet assembly 50. Some players might prefer a pad 54 that extends downward farther toward the approximate center of the cheek below the target area in addition to a pronounced depression of the target area

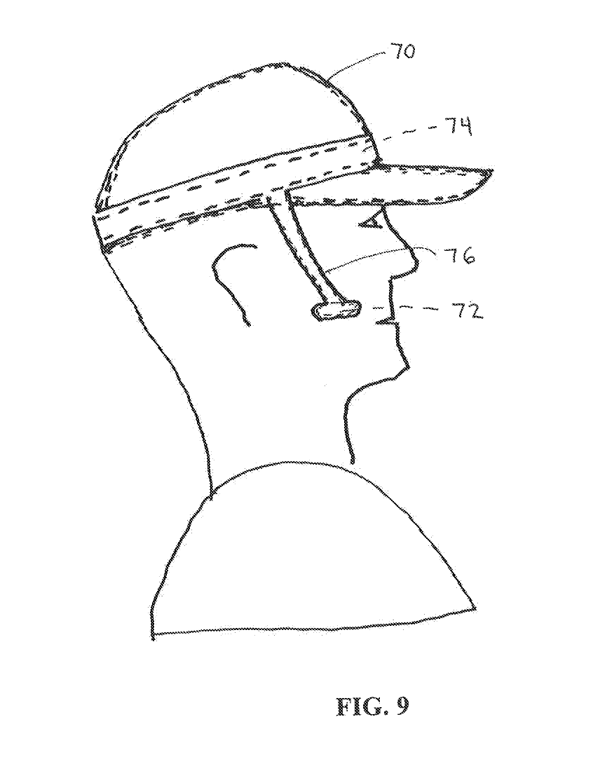

FIG. 9 shows user another embodiment of the present invention including a cap assembly 70 including pads 72 held tightly in place against the target area of the cheek via spring frame 74 and spring leg 76. Cap assembly 70 may be a baseball-style cap.

Spring frame 74 and spring leg 76 in this embodiment are constructed of a spring material, such as stainless steel, which will hold its shape and exert generally constant pressure inwardly thereby directing forces into the target area of the user. Portions of cap assembly 70 may be constructed of fabric, such as a heavy duty cotton/polyester weave. The pair of spring legs 76 have pads 72 affixed to each end. Pads may be constructed of molded medical grade silicone. The inner surface (facing user) of pads 72 is somewhat rounded or convex. Spring leg 76 can be curved inward at its lower section in such a way that pad 72 can be arranged and positioned to provide a somewhat angular upward force against the target area of the cheek in addition to an approximately horizontal force applied inward against the target region. In some users a slight upward angle to the force applied provides somewhat better breathing than a straight horizontally applied force. Double sided medical grade tape tabs can be constructed of the proper size and applied to the cheek side surface of pads 72 to help keep pads 72 in proper position during vigorous exercise.

Stainless steel alloy is a reasonable choice for compactness, durability and rebound memory and is selected for this embodiment, but many other materials could be used to construct spring frame 74 and spring leg 76 including other types of metals, plastics or composites providing higher strength, better rebound memory and light weight. Spring frame 74 is preferably constructed of a spring type material with good rebound memory, but it is also possible to construct frame 74 of a mostly rigid material and provide compressible pads with rebound memory instead. Spring legs 76 may be provided with a telescoping adjustment means.

A cap can also be constructed to include larger cheek depressors targeted to the target area in order to decrease an air accumulator effect which may occur during hard respiration of some when they engage in vigorous exercise involving maximum exertion and hard respiration. For some a larger cheek pad may provide more optimal breathing than a smaller one. In order to provide for a larger pad 72 or increased three for pad 72 against the target area of the user's cheek, a stronger spring leg 76 can be used to provide more spring force inward.

In order to don cap assembly 70, a user spreads apart the spring legs 76, places the cap in proper wearing position, and then releases the pair of spring legs 76, allowing the pair of pads 72 to move inwardly to press snuggly and depress into the target area of each cheek. Legs 76 can be adjusted by increasing or decreasing their length by use of the telescoping feature built into each leg 76 (not shown but understood in the art) if necessary for good contact between the pad 72, and the target area of the cheek. A double sided medical grade tape tab can be applied to the inner surface of each of the pair of pads 72, which in addition to the tension provided by spring leg 76 will help keep pad 72 in proper position during hard or jarring exercise and will help keep the cap in place. The double sided tape tabs are disposable and can be replaced at each use of the hat or as frequently as desired by the user.

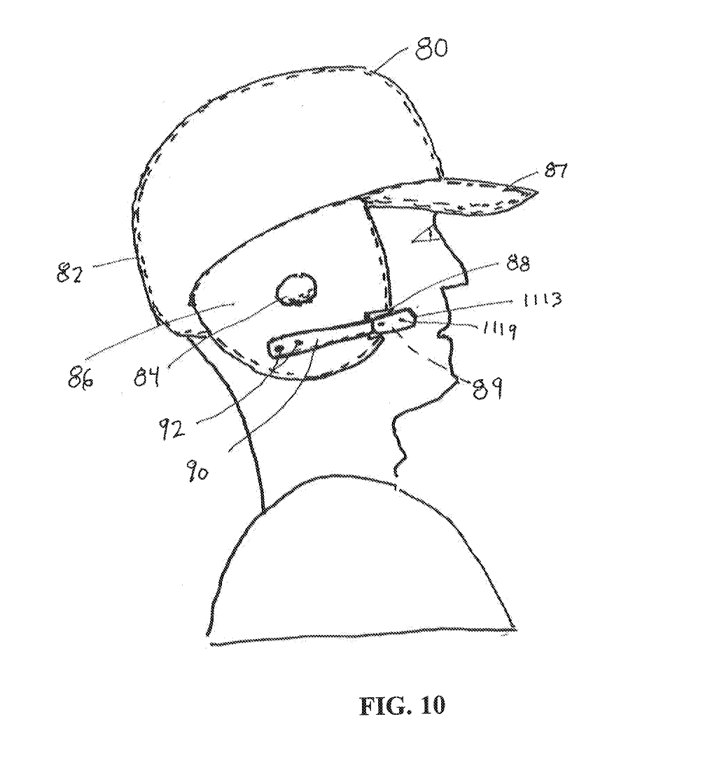

FIG. 10 depicts another embodiment of the invention. A batting helmet assembly 80 is a protective helmet used by baseball players. Batting helmet assembly 80 has a shell 82 which contains an inner padding (not shown) which helps cushion and protect the head from shock when hit by a baseball. An aperture 84 is provided to ear protector cavity 86 to allow the wearer to hear. Batting helmet assembly 80 is typically constructed of a strong resilient type of molded plastic or fiberglass, but carbon fiber can be selected for molding as an even stronger, lighter option. Batting helmet assembly 80 is provided with a visor 87 and a cutaway 88, which is a small cutaway from the bottom front of ear protector cavity 86. Cutaway 88 provides clearance for the penetration of a pad 89 through ear protector cavity 86 to make good contact with the target area of the cheek of the user,

Ear protector cavity 86 is provided with a spring 90 to which pad 89 is provided to its inward facing side toward the user's face, Pad 89 is molded of medical grade silicone which can feel sticky to the touch when cleaned with soapy water, Pad 89 is somewhat convex at its inner side which faces the cheek and has no sharp edges. Spring 90 is comprised in this instance of a high quality stainless steel which provides good strength and rebound memory while providing adequate force against pad 89 to depress the target area of the cheek of the user,

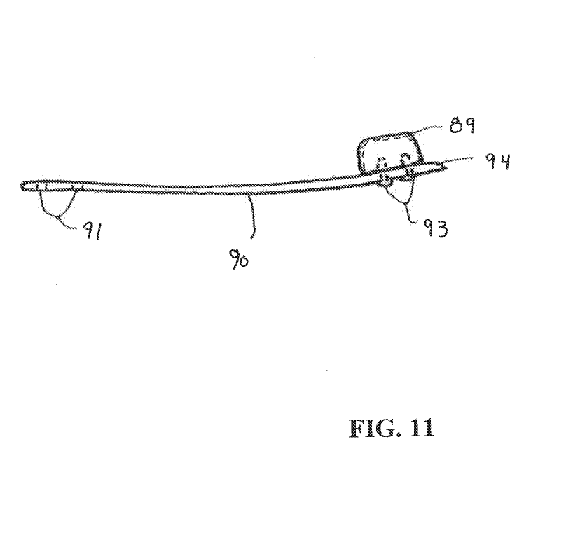

As shown in FIG. 11, spring 90 is provided with apertures 91 at each end. These end apertures 91 are used to attach spring 90 to ear protector cavity 86 via, for example, rivets 92 passing through holes provided to shell 82 (not shown), Threaded fasteners 93 are used to secured pad 89 to spring 90. Other types of fasteners and even glue pads or glue can also be used to affix pad 89 to spring 90. Pad 89 can be provided in different widths in order to provide for individual differences in facial anatomy, thereby insuring proper tension to spring 90 which will in turn provide proper depression of the target area of the cheek. Tab 94 is provided at one end of spring 90 to provide a point for the user to grab and manipulate pad 89 away from the target area of the cheek, so that the helmet can be put on or removed.

As shown in FIG. 10, batting helmet assembly 80 positions pad 89 against the target area of the cheek via. spring 90. The integral use of a cheek depressor to a batting helmet assembly 80 allows enhanced breathing for the batter. Batting helmet assembly 80 can be donned or removed by grasping tabs 94 and pulling each spring 90 outward to thereby pull each pad 89 away from the cheek of the user.

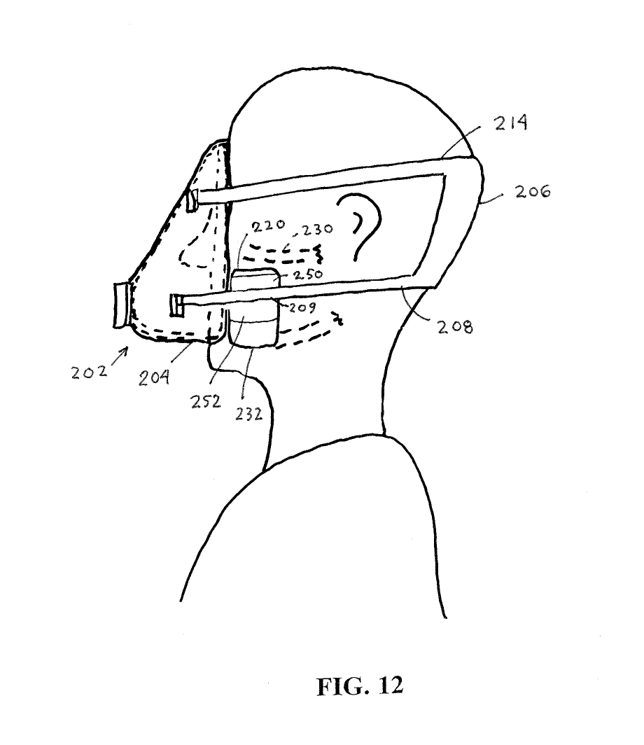

FIG. 12 depicts another embodiment of a CD assembly having pad 200 providing compression of cheek. tissue under the cheek bone (zygomatic bone) and above the jaw bone. Pad 200 is compressed against the cheek but without abrading, bruising or harming the teeth gums or cheek. A PAP mask assembly 202 is depicted in proper arrangement and position.

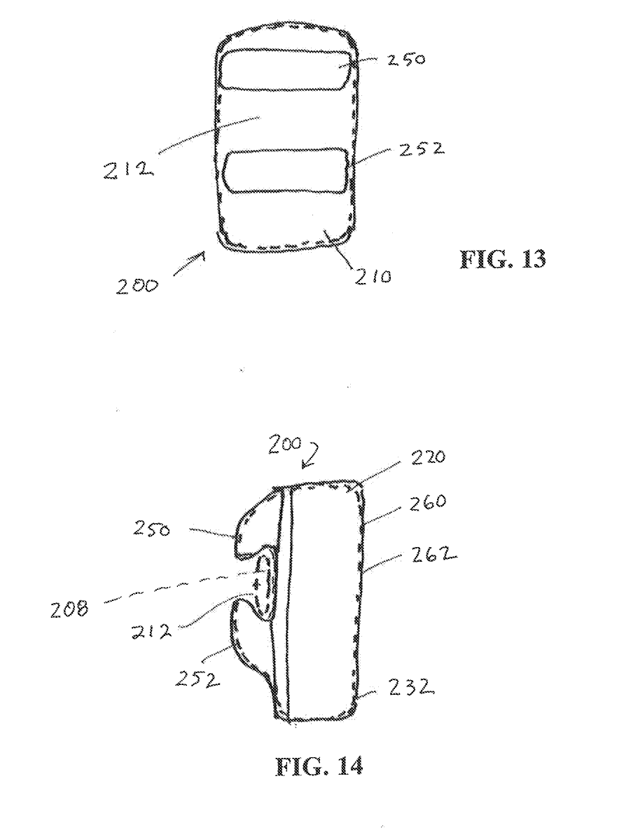

Mask assembly 202 is a full mask type assembly of the type typically used in PAP type therapy and has a flexible silicone seal 204, with said seal fitted against the face of the user to prevent significant air leakage while air is being delivered under pressure by a PAP type unit to mask assembly 202. Mask assembly 202 has an attachable and removable headgear assembly 206 connected to it. Headgear assembly 206 has an integral lower headgear strap 208 which is arranged and positioned to pass through and be restrained in headgear strap channel 209 wherein pad 200 is positioned to permit such an arrangement and positioning of lower headgear strap 208 across top plate 210 and into and through headgear strap channel 212 (shown in FIG. 13). Headgear assembly 206 includes an upper headgear strap 214 which is attachable to the upper part of mask assembly 202.

As shown in FIG. 12, pad 200 is arranged and positioned such that the upper face side 220 of pad 200 makes contact beneath the zygomatic bone 230 and the lower face side 232 of pad 200 makes contact with above the lower mandible 236. This particular arrangement and positioning of pad 200 allows it to be cradled and supported by lower headgear strap 208 to thereby provide stability for the assembly while it is being used during PAP type therapy. This arrangement and positioning depicted in FIG. 12 also can provide some side support for a portion of seal 204 of face mask assembly 202 if properly arranged and positioned up against it on the cheek side of seal 204, thereby helping to provide for good and reliable sealing at that point of contact between seal 204 and the CD assembly. The compressibility of pad 200 to conform to the morphology and structure of the side of the face where it is placed, optimally providing light contact between the cheek with the teeth and gums, can provide stability, comfort and effective function of the cheek depressor by allowing the contact of the compressed pad against the cheek to provide adequate depression of the cheek without abrading, bruising or harming the gums, teeth or inside of the cheek, thus preventing significant cheek movement from the depressed position during exhalation. When suitably arranged and positioned, the CD assembly can depress the cheeks effectively to reduce or eliminate a significant air accumulator effect in the mouth cavity of the user which occurs when the cheeks are not depressed during the use of a PAP type unit.

FIGS. 13-14 illustrate one embodiment of cheek depressing pad 200. Use of strap retainer 250 and strap retainer 252 affixed to top plate 210 provides a headgear strap channel 212 which will contain and hold a lower headgear strap 208, while at the same time allowing lower headgear strap 208 to be adjusted or positioned whereby it can be pulled back and forth or through headgear strap channel 212 (FIG. 14), or pulled out of or placed into headgear channel 212, while otherwise being able to hold lower headgear strap 208 stable when not being adjusted or positioned. Pad 200 has a face side 260 for contact with the face of the device user as shown in FIG. 14. FIG. 14 shows pad 200 to have an upper face side 220 and a lower face side 232, and a pad center 262, which are compressible against a cheek bone (just below zygomatic bone) 230 and above the jawbone (mandible) 236 of a user when the CD assembly is properly arranged and positioned.

FIG. 15 shows another embodiment of cheek-engaging pad 200 as it will typically appear in approximate compressed form when cradled under the cheekbone (zygomatic bone) and above the jaw bone. This depiction is an approximation and any actual compression of pad 200 may vary somewhat from this without departing from this novel art. FIG. 15 shows an estimated side view depiction of pad 200 with an uncompressed outline 270 of pad 200 shown in comparison to a compressed outline 272 of pad 200. Compressed outline 272 is the result of placing the CD assembly in proper arrangement and position in relation to lower headgear strap 208, to facemask assembly 202 and to the user (see FIG. 12) with proper tensioning of lower headgear strap 208.

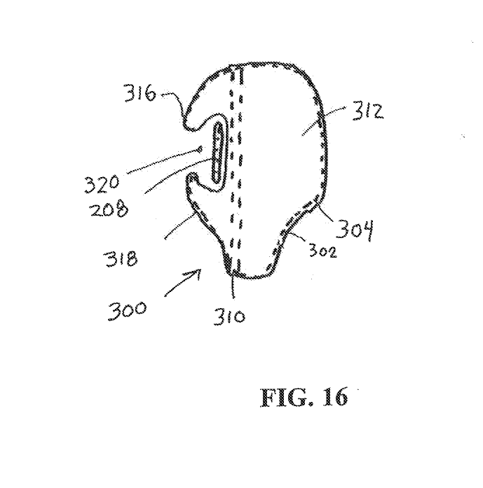

The use of a pre-molded integral cheek depressor such as CD assembly 300 of FIG. 16 is an alternative way to provide a cheek depressor, one which has an integral pad perimeter such as that shown in FIG. 16 as pad perimeter 302. Because pad perimeter 302 is pre-formed it can make contact under the cheek bone and upon the jaw bone to provide a suitable and yet comfortable support for a pad center which is thicker, such as pad center 304, which is centered to the approximate center of the cheek for depressing the cheek. Use of this pre-molded bi-level CD assembly 300 of FIG. 16 can provide good depression of the user's cheek in an overall thinner unit. When CD assembly 300 is properly arranged and positioned, CD assembly 300 prevents or minimizes an air accumulator effect which can occur without the cheeks being effectively depressed.

The CD assembly 300 of FIG. 16 includes support plate 310, which can be comprised of a single piece of molded plastic, composite, sheet metal or any other appropriate substance which is free of sharp corners and/or burrs which could wear through or cut through the foam material molded around it, CD assembly 300 is formed with a single piece of foam of plastic or rubber type molded around this support plate by using a mold as is known in the art of plastic or rubber molding, using a material which will be somewhat compressible when properly arranged and positioned on a user. In this case, after CD assembly 300 has been molded it is dipped in a thin plastic compound to provide a smooth sealed surface coating as is also known in the art. CD assembly 300 has a pre-molded integral bi-level pad 312, is provided with a pad perimeter 302 for contact under the cheek bone (zygomatic bone) and above the jaw bone (mandible), and will be placed at an approximate cheek center.

Pad center 304 provides for depressing the cheek effectively and keeping it depressed while, for example, a PAP type unit is operated without causing abrading, bruising or damage to the cheek, gums or upper or lower jaw. CD assembly 300 has a headgear strap retainer 316 and a headgear strap retainer 318 also molded integral to it, thus providing a headgear strap channel 320 for a lower headgear strap 208 of FIG. 12, in this way, lower headgear strap 208 of FIG. 12 can be retained securely while the user loosens or adjusts lower headgear strap 208 or removes lower headgear strap 208 from mask assembly 202 in order to remove mask assembly 202 of FIG. 12. This allows removal, storage or re-fitting of mask assembly 204 by and from user without lower strap 208 coming loose from headgear strap channel 320 unless the user chooses to pull it out of headgear strap channel 320. Headgear strap channel 320 allows the easy adjustment of the lower strap during use.

Other embodiments of a cheek depressor may include semi-spherical elements that are coupled to one or more straps of a PAP mask. The semi-spherical elements may include portions of different compressibility to improve fit or efficacy of the cheek depressor. The semi-spherical cheek depressors can be solid or liquid filled.

CD assembly 300 can be custom molded to an impression taken of the face using any of a number of available means, including but not limited to compressible foam plastic non rebounding molding such as that used for making impressions of the bottom side of the foot by orthotic technicians and podiatrists. CD assembly 300 can alternatively be pre molded in several distinct prearranged standard sizes thus allowing the individual selection of a pre-molded CD assembly 300 at the time of fitting to avoid any waiting times for a custom molded order. Either method, premade standard sizes or a custom molded method, can provide for a very good and very comfortable fit without much compression of the pad 312 needed, thus allowing pad perimeter 302 and pad center 304 to be thinner than otherwise possible. And it is also possible to pre-mold and/or construct pad 312 in different stiffnesses, such as a somewhat thinner stiffer pad perimeter 302 and a thicker, softer, somewhat more compressible pad center 304 in order to provide pad center 304 with a mildly compressible but thicker foam for maximum comfort at the cheek when it is depressed.

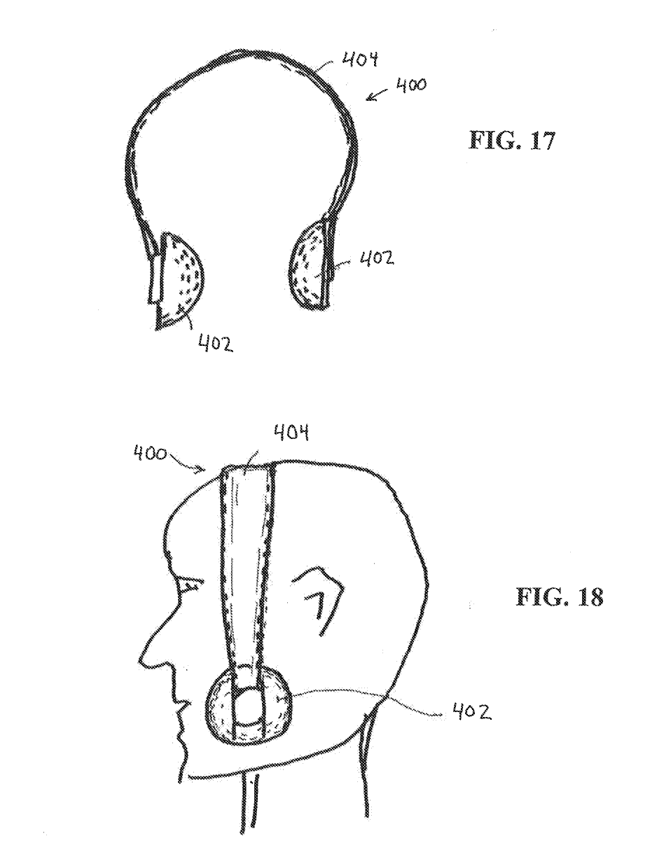

FIG. 17 is a front view of another embodiment of the invention, a CD assembly 400. CD assembly 400 is comprised of a pair of molded silicone pads 402, which are suitably fastened to the bottom ends of a yoke 404 which ideally should have spring like properties being able to rebound to its starting shape While holding tension when displaced to a more open position. In this case yoke 404 has an outside cover constructed of a nylon plastic type compound to which a formed and pre-stressed steel spring is molded inside (not shown). The pair of pads 402 can be suitably attached to the ends, one each, of yoke 404 by insertion into orifices molded into the pair of pads 402. There are no sharp edges anywhere on yoke 404 or either of the pair of pads 402. CD assembly 400 is placed in proper position over the head and each of the pair of pads 402 is adjusted by moving them up or down on yoke 404 to place them at proper position of the approximate center of each cheek. Proper thickness of the pair of pads 402 can be selected to provide adequate tension against each cheek. It is also easy to manufacture this freestanding embodiment as a very simple singly molded piece out of a plastic composite with good support strength, spring characteristics and memory. Carbon fiber, which has many benefits such as strength and lightness, can also be used.

FIG. 18 shows CD assembly 400 properly placed and positioned on a patient's head. Yoke 404 may be constructed of various desirable high tensile strength metal alloys or plastic compounds which are light and comfortable to use. Of course a pad can be arranged at the sides and/or top of yoke 404 to increase the comfort of the patient.

The actual CDs can be made very compact for easy application to use by musicians or those engaging in various sports, organized and individual, which may include exercising, jogging or swimming. This embodiment can be custom made to provide a very compact streamlined fit and still be able to enhance exhalation for musicians or respiration in general for those engaging in physical exercise. The CD assembly of this embodiment may prove over time to be useful in various professional and amateur sports as an enhancement to conditioning and performance. This embodiment of the CD assembly offers great flexibility for a wide variety of applications, medical and non medical including but not limited to individual and team sports, the playing of musical instruments powered by exhalation and other uses which will utilize the benefits of enhanced inhalation and/or exhalation. For some individual users, the CD assembly must not necessarily be placed at the approximate center of each cheek to attain a benefit of enhanced inhalation and/or exhalation. For example some individuals seem to respond well when the CD assembly is placed at the upper part of the cheeks and the pads are of smaller size.

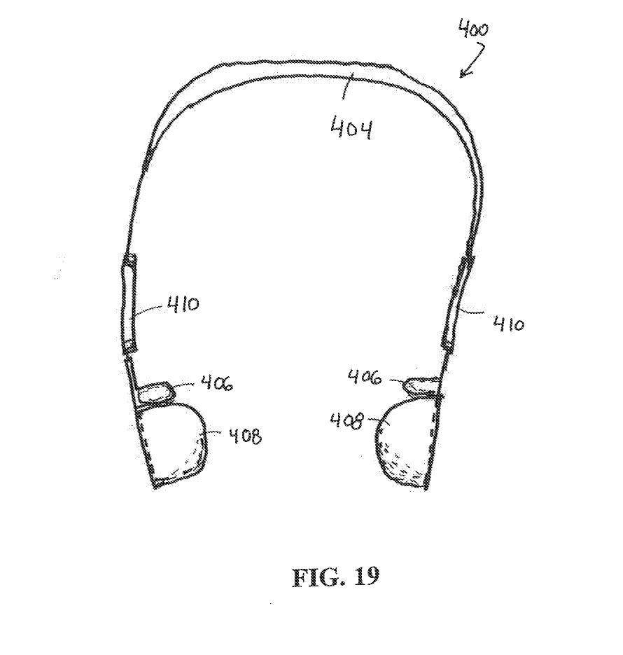

FIG. 19 illustrates another embodiment of the present invention including a telescoping yoke 400 with a pair of pad assemblies 402 secured at ends of yoke 400. Pad assembly 402 includes a pair of pad portions 406, 408. Pad portion 406 can be substantially softer (more compressible) than pad portion 408. Pad portion 408 is optimally positioned at the target area of the user. Telescoping couplers 410 are provided to adjust the fit of yoke 400.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A patient interface device for depressing a targeted portion of a user's cheek against underlying teeth structure to enhance user respiration comprising:
a pad adapted to engage a target area of the user's cheek and maintain cheek tissue at the target area against an underlying tooth structure during inhalation and exhalation, said target area being located between above a mandible and below a zygomatic bone of the patient, and with the pad engaging the target area with a pad force of between 0.5 pounds to 3 pounds, and with substantially no external force being applied by the device to the zygomatic bone or mandible of the user; and
a head support structure coupled to the pad and maintaining the pad at the target area during a therapy session.

2. The device of claim 1 wherein the head support structure includes a PAP mask.

3. The device of claim 1 wherein the head support structure includes is a spring.

4. The device of claim 3 wherein the spring is a yoke structure.

5. The device of claim 1 wherein said pad includes at least two portions having at least two substantially different levels of compressibility.

6. The device of claim 1 further comprising an adhesive for securing the pad in the target area,

7. The device of claim 6 wherein the adhesive is provided by an adhesive tape.

8. The device of claim 1 wherein the pad is a semi-spherical body.

9. The device of claim 1 wherein the pad force is between about 0.5 pounds to 1.0 pounds to the target area.

10. The device of claim 1 wherein the pad is gas-filled.

11. A patient interface device comprising:
a pair of pads adapted to be placed against a patient's cheeks, with each of said pair of pads being placed at a target area above a jawbone and a below a zygomatic bone of the patient; and
a head-worn support structure include a spring depressing the pair of pads against said patient's cheek tissue, said spring providing a cheek tissue-depressing force to said pair of pads resulting in an application of an external force to a patient's cheek tissue only at the target area away from both the mandible and the zygomatic bone of the patient, said force tending to limit movement of the patient's cheek tissue during respiration and maintain inner cheek tissue against the patient's gums and teeth during use, and with substantially no external forces being applied by the spring to either the zygomatic hone or mandible of the patient.

12. The device of claim 11 wherein the support structure includes a PAP mask.

13. The device of claim 11 wherein said pad includes at least two portions having at least two substantially different levels of compressibility.

14. The device of claim 11 further comprising an adhesive for securing the pad in the target area.

15. The device of claim 11 wherein the pad force is between about 0.5 pounds to 1.0 pounds to the target area.

16. The device of claim 11 wherein the pad is gas-filled.

17. A method using a patient interface device comprising:
providing a pad against a user's cheek tissue at a target area between a jawbone and a zygomatic bone of the user;
securing said pad against the user's cheek tissue with an external structure, said external structure applying a force through the pad to the user's cheek only at the target area and away from both the jawbone and zygomatic bone of the user; and
restraining movement of the user's cheek tissue by application of said force to said device, with said restraining limiting cheek tissue movement by maintaining inner cheek tissue against gums and teeth of the user thereby enhancing respiration of the user.

18. A method of using an interface device comprising:
providing a pad in contact with a targeted portion of the user's cheek tissue located above a jawbone and below a zygomatic bone of the user;
securing the pad in place with an external structure, with said external structure providing a force through said pad to maintain contact between the user's inner cheek and teeth and gums only at the targeted area and with substantially no external forces being applied by the external structure to either the mandible or zygomatic bone of the user; and
restraining movement of the user's cheek tissue by application of said force at the targeted area.
